(19) United States
(12) Patent Application Publication
(10) Pub. No.: US 2016/0270755 A1
(43) Pub. Date: Sep. 22, 2016

(54) RADIATION IMAGING APPARATUS AND RADIATION IMAGING SYSTEM

(71) Applicant: CANON KABUSHIKI KAISHA, Tokyo (JP)

(72) Inventors: Katsuro Takenaka, Honjo-shi (JP); Shinichi Takeda, Honjo-shi (JP); Atsushi Iwashita, Saitama-shi (JP); Sho Sato, Saitama-shi (JP); Kosuke Terui, Honjo-shi (JP); Yoshiaki Ishii, Honjo-shi (JP)

(21) Appl. No.: 15/064,757

(22) Filed: Mar. 9, 2016

(30) Foreign Application Priority Data
Mar. 20, 2015 (JP) 2015-058292

(51) Int. Cl. A61B 6/00; G01T 1/20

(52) U.S. Cl. CPC A61B 6/5258; G01T 1/20; A61B 6/54; A61B 6/4233

(57) ABSTRACT

A radiation imaging apparatus is provided. The apparatus comprises a plurality of pixels and a signal processing unit configured to read out an analog signal from each pixel and output an image signal. The signal processing unit comprises a conversion unit configured to convert the analog signal into a digital signal using an A/D converter such that the digital signals of a first group pixels include a first offset components and the digital signals of a second group pixels include a second offset components, and a digital signal processing unit. The digital signal processing unit calculates a correction value using the digital signals of the first and the second group pixels, and performs correction of reducing an influence caused by the A/D converter in the digital signals of the first group pixels using the correction value, thereby generating the image signal.

### BACKGROUND OF THE INVENTION

1. Field of the Invention

The present invention relates to a radiation imaging apparatus and a radiation imaging system.

2. Description of the Related Art

In recent years, a radiation imaging apparatus using a flat panel detector formed using a semiconductor material has been put into practical use as an imaging apparatus used for medical image diagnosis or nondestructive inspection. Such a radiation imaging apparatus includes an A/D converter that converts an analog signal generated by the detector into a digital signal. However, concerning the conversion characteristic between the input analog signal and the output digital signal, the A/D converter may have non-linearity instead of exhibiting ideal linearity. Japanese Patent Laid-Open No. 2010-141716 discloses a radiation imaging apparatus that performs different change processing for an analog signal on a column basis and then inputs the analog signal to an A/D converter, or performs processing of changing the conversion characteristic of the A/D converter on a column basis and then converts an analog signal into a digital signal. With this processing, new output differences are generated between digital signals output in the row direction, and an output difference caused by the conversion characteristic of the A/D converter becomes unnoticeable. This reduces a visual influence on a captured image.

### SUMMARY OF THE INVENTION

In the arrangement disclosed in Japanese Patent Laid-Open No. 2010-141716, however, when different processing is performed on a column basis, a new stripe-shaped artifact is generated on a column basis by the conversion characteristic of the A/D converter.

An aspect of the present invention provides a technique of reducing the stripe-shaped artifact and suppressing degradation in the quality of a captured image caused by the non-linearity of the conversion characteristic of an A/D converter.

According to some embodiments, a radiation imaging apparatus comprising: a plurality of pixels arranged in a matrix and configured to detect radiation; and a signal processing unit configured to read out an analog signal from each pixel and output an image signal, wherein the signal processing unit comprises a conversion unit configured to convert the analog signal from each pixel into a digital signal using an A/D converter such that the digital signals of pixels included in a first group include offset components of a first value and the digital signals of pixels included in a second group include offset components of a second value different from the first value, and a digital signal processing unit configured to process the digital signal and output the image signal, and wherein the digital signal processing unit calculates a correction value using the digital signals of the pixels included in the first group and the digital signals of the pixels included in the second group, and performs correction of reducing an influence caused by a conversion characteristic of the A/D converter in the digital signals of the pixels included in the first group using the correction value, thereby generating the image signal, is provided.

According to some other embodiments, a radiation imaging system comprising a radiation imaging apparatus and a radiation generating apparatus, wherein the radiation imaging apparatus comprises a plurality of pixels arranged in a matrix and configured to detect radiation, and a signal processing unit configured to read out an analog signal from each pixel and output an image signal, the signal processing unit comprises a conversion unit configured to convert the analog signal from each pixel into a digital signal using an A/D converter such that the digital signals of pixels included in a first group include offset components of a first value and the digital signals of pixels included in a second group include offset components of a second value different from the first value, and a digital signal processing unit configured to process the digital signal and output the image signal, the digital signal processing unit calculates a correction value using the digital signals of the pixels included in the first group and the digital signals of the pixels included in the second group, and performs correction of reducing an influence caused by a conversion characteristic of the A/D converter in the digital signals of the pixels included in the first group using the correction value, thereby generating the image signal, and the radiation generating apparatus is configured to generate radiation, is provided.

Further features of the present invention will become apparent from the following description of exemplary embodiments (with reference to the attached drawings).

### BRIEF DESCRIPTION OF THE DRAWINGS

FIG. 1 is a block diagram of a radiation imaging apparatus according to the present invention;

FIG. 2 is an equivalent circuit diagram of the radiation imaging apparatus according to the present invention;

FIGS. 3A and 3B are graphs showing the input/output characteristic of the A/D converter of the radiation imaging apparatus according to the present invention;

FIGS. 4A to 4D are graphs showing the input/output characteristic of the A/D converter of the radiation imaging apparatus according to the present invention;

FIG. 5 is a timing chart showing the driving timing of the radiation imaging apparatus according to the present invention;

FIGS. 6A to 6C are views showing the step difference correction method of the radiation imaging apparatus according to the present invention;

FIGS. 7A to 7C are views showing the step difference correction method of the radiation imaging apparatus according to the present invention;

FIGS. 8A to 8C are views showing the step difference correction method of the radiation imaging apparatus according to the present invention; and

FIGS. 9A and 9B are conceptual views of a radiation imaging system using the radiation imaging apparatus according to the present invention.

### DESCRIPTION OF THE EMBODIMENTS

A detailed embodiment of a radiation imaging apparatus according to the present invention will now be described with reference to the accompanying drawings. Note that in the following description and drawings, common reference numerals denote common components throughout a plurality of drawings. Hence, the common components will be described by cross-referring to the plurality of drawings, and a description of components denoted by common reference numerals will appropriately be omitted. Note that radiation according to the present invention can include not only α-rays, β-rays, and γ-rays that are beams generated by particles (including photons) emitted by radioactive decay but also beams having energy equal to or higher than the energy of these beams, for example, X-rays, particle beams, and cosmic rays.

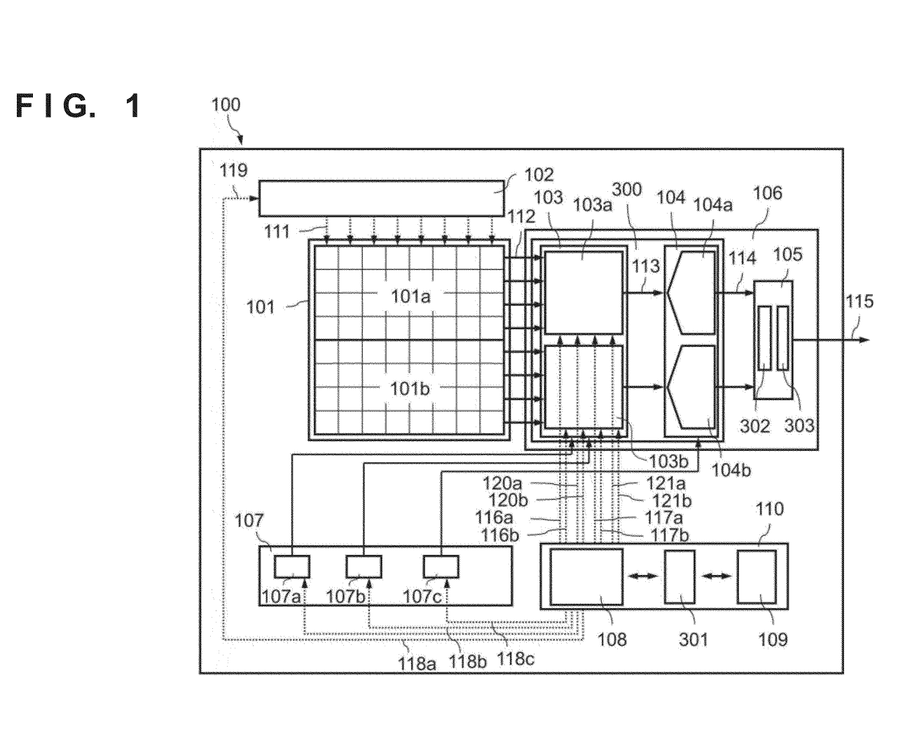

FIG. 1 is a block diagram conceptually showing the arrangement of a radiation imaging apparatus 100 according to this embodiment. The radiation imaging apparatus 100 shown in FIG. 1 includes a detection unit 101, a driving circuit 102, a signal processing unit 106, a power supply unit 107, and a control unit 110. The detection unit 101 includes a plurality of pixels arranged in a matrix and configured to convert radiation or light into an analog signal and detect the radiation. In FIG. 1, pixels arranged in the horizontal direction will be referred to as a pixel column, and pixels arranged in the vertical direction will be referred to as a pixel row. The driving circuit 102 scans the plurality of pixels provided in the detection unit 101 and drives the detection unit 101 to output analog signals from the detection unit 101. In this embodiment, for the sake of simplicity, the detection unit 101 includes pixels of 8 rows×8 columns which are divided into a first pixel group 101a and a second pixel group 101b each including four pixel columns.

An analog signal 112 output from the detection unit 101 is input to the signal processing unit 106. The signal processing unit 106 includes a conversion unit 300 including a readout circuit 103 and an A/D converter 104, and a digital signal processing unit 105. The analog signals 112 output from the first pixel group 101a are input to the conversion unit 300 and read out by a first readout circuit 103a. An analog signal 113 output from the first readout circuit 103a is input to a first A/D converter 104a, converted into a digital signal 114, and output from the conversion unit 300. Similarly, the analog signals 112 output from the second pixel group 101b are read out by a second readout circuit 103b, input to a second A/D converter 104b, and converted into the digital signal 114. The digital signal 114 output from the A/D converter 104 is input to the digital signal processing unit 105. The digital signal processing unit 105 includes a correction value calculation unit 302 that calculates, for the input digital signal 114, a correction value to reduce the influence of the conversion characteristic of the A/D converter 104, and a correction unit 303 that corrects the digital signal using the correction value. The digital signal processing unit 105 performs simple digital signal processing such as digital multiplexing processing or offset correction, and generates and outputs an image signal 115. When the image signal 115 is output from the radiation imaging apparatus 100, the captured image can be observed on an external display (not shown) or the like.

The power supply unit 107 gives, to the signal processing unit 106, reference voltages serving as biases corresponding to the circuits in the signal processing unit 106. The power supply unit 107 includes a first power supply unit 107a and a second power supply unit 107b each of which gives a reference voltage to the readout circuit 103, and a third power supply unit 107c that gives a reference voltage to the A/D converter 104.

The control unit 110 includes a control circuit 108, a storage unit 109, and an offset generation unit 301. The control circuit 108 controls the driving circuit 102, the signal processing unit 106, and the power supply unit 107, and performs a captured image readout operation. The storage unit 109 stores information about the non-linearity of the conversion characteristic between the analog signal input to the A/D converter 104 and the digital signal output from the A/D converter 104. The offset generation unit 301 controls at least one of the signal processing unit 106 or the power supply unit 107. In this case, the offset generation unit 301 may control at least one of the signal processing unit 106 or the power supply unit 107 based on the information in the storage unit 109. The control circuit 108 and the offset generation unit 301 may synchronize and control the radiation imaging apparatus 100. The control unit 110 supplies a first reference voltage adjusting signal 118a, a second reference voltage adjusting signal 118b, and a third reference voltage adjusting signal 118c to the first power supply unit 107a, the second power supply unit 107b, and the third power supply unit 107c, respectively. The control unit 110 also supplies a gain adjusting signal 116, a sample hold control signal 120, a multiplexing control signal 117, and a setting signal 121 for a D/A converter to the readout circuit 103. In FIG. 1, “a” is added to the reference numeral of each signal supplied to the readout circuit 103a out of the readout circuit 103, and “b” is added to the reference numeral of each signal supplied to the readout circuit 103b. The control unit 110 further supplies a driving control signal 119 to the driving circuit 102, and the driving circuit 102 supplies a driving signal 111 to the detection unit 101 based on it.

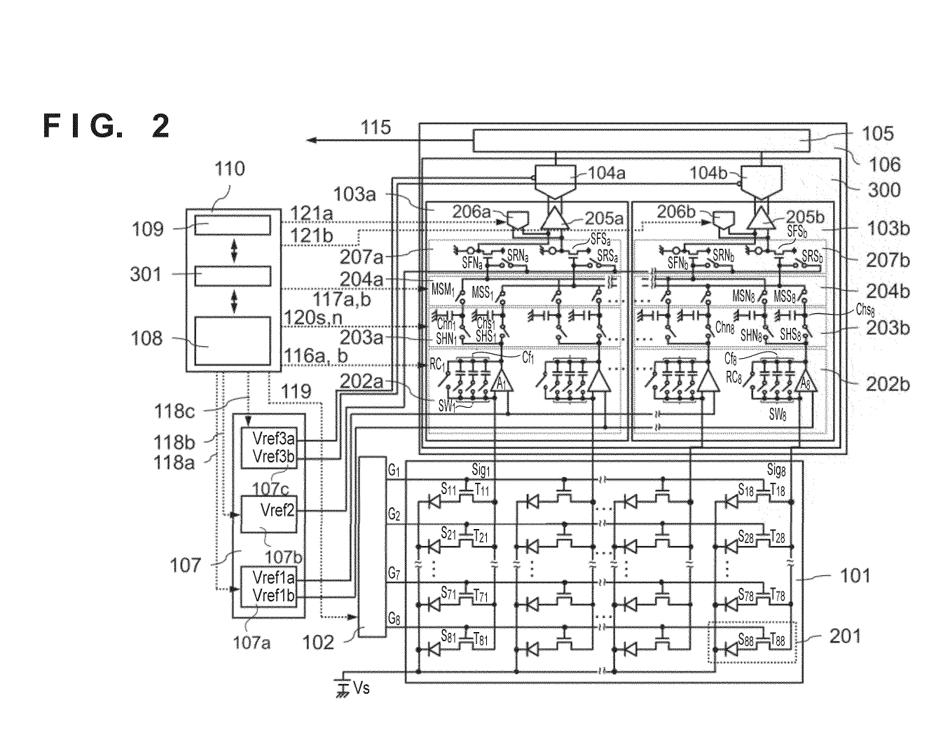

FIG. 2 is a conceptual equivalent circuit diagram of the radiation imaging apparatus 100 according to this embodiment. The detection unit 101 includes a plurality of pixels 201 arranged in a matrix. In FIG. 2, pixels arranged in the horizontal direction will be referred to as a pixel row, and pixels arranged in the vertical direction will be referred to as a pixel column. In this embodiment, 8×8 pixels 201 are arranged on 8 rows×8 columns. The pixel 201 on the ith row and jth column includes a conversion element Sᵢⱼ that converts radiation or light into charges, and a switching element Tᵢⱼ that outputs the analog signal 112 that is an electrical signal corresponding to the charges. In the following explanation, the conversion elements Sij will generically be referred to as a conversion element S, and the switching elements Tᵢⱼ will generically be referred to as a switching element T. As the conversion element S that converts light into charges, a photoelectric conversion element such as a PIN photodiode that is mainly made of amorphous silicon and arranged on an insulating substrate such as a glass substrate may be used. As the conversion element that converts radiation into charges, an indirect conversion element including a wavelength converter configured to convert radiation into light in a wavelength band sensible by a photoelectric conversion element or a direct conversion element configured to directly convert radiation into charges may be used. As the switching element T, a transistor including a control terminal and two main terminals may be used. If the photoelectric conversion element is a pixel arranged on an insulating substrate, a thin film transistor (TFT) may be used. One electrode of the conversion element S is electrically connected to one of the two main terminals of the switching element T, and the other electrode is electrically connected to a bias power supply Vs via a common wire. The switching elements of the plurality of pixels 201 in the row direction are controlled via driving wires G₁ to G₈ arranged for the respective rows. For example, the control terminals of switching elements T₁₁ to T₁₈ are commonly electrically connected to the driving wire G₁ of the first row. The driving circuit 102 gives a driving signal that controls the ON state of a switching element via the driving wire on a row basis. The switching elements, for example, switching elements T₁₁ to T₈₁ of the plurality of pixels 201 in the column direction each have the other main terminal electrically connected to a signal wire Sig₁ of the first column. In the ON state, the switching elements T₁₁ to T₈₁ each output an electrical signal corresponding to the charges of the conversion element to the readout circuit 103 via the signal wire. A plurality of signal wires Sig₁ to Sig₈ arranged in the column direction transmit the analog signals 112 output from the plurality of pixels 201 of the detection unit 101 to the readout circuit 103 in parallel. In this embodiment, the detection unit 101 is divided into the first pixel group 101a and the second pixel group 101b each including four pixel columns. The electrical signals output from the first pixel group 101a are read out by the first readout circuit 103a in parallel. The electrical signals output from the second pixel group 101b are read out by the second readout circuit 103b in parallel.

Each readout circuit 103 includes an amplification circuit unit 202, a sample hold circuit unit (to be referred to as an SH circuit unit hereinafter) 203, a multiplexor 204, an output buffer 207, a variable amplifier 205, and a D/A converter 206. In FIG. 2, “a” is added to the reference numeral of each constituent element included in the readout circuit 103a out of the readout circuit 103, and “b” is added to the reference numeral of each constituent element included in the readout circuit 103b. The electrical signals in parallel output from the first pixel group 101a and the second pixel group 101b are input to the first readout circuit 103a and the second readout circuit 103b and first amplified by a first amplification circuit unit 202a and a second amplification circuit unit 202b. Each of the first amplification circuit unit 202a and the second amplification circuit unit 202b includes, for each signal wire, an amplification circuit including an operational amplifier A that amplifies and a readout electrical signal and outputs it, an integral capacitor group Cf, a switch group SW configured to switch the amplification factor, and a reset switch RC configured to reset the integral capacitors. The analog signal 112 output from the detection unit 101 is input to the inverting input terminal of the operational amplifier A, and the amplified electrical signal is output from the output terminal. In this embodiment, the first power supply unit 107a inputs a reference voltage Vref1a to the non-inverting input terminal of each amplification circuit of odd-numbered columns, and inputs a reference voltage Vref1b to the non-inverting input terminal of each amplification circuit of even-numbered columns. The reference voltage Vref1a and the reference voltage Vref1b may have the same value or values different from each other. The integral capacitor group Cf including a plurality of integral capacitors arranged in parallel is arranged between the inverting input terminal and the output terminal of the operational amplifier A.

Next, the electrical signals amplified by the first amplification circuit unit 202a and the second amplification circuit unit 202b are input to a first SH circuit unit 203a and a second SH circuit unit 203b each configured to sample and hold an electrical signal. Each of the first SH circuit unit 203a and the second SH circuit unit 203b includes, for each amplification circuit, a sample hold circuit formed from a noise sampling switch SHN and a signal sampling switch SHS, and a noise sampling capacitor Chn and a signal sampling capacitor Chs. Each switch of the SH circuit unit 203 is controlled by the sample hold control signal 120 from the control unit 110. Next, the electrical signals in parallel read out from the first SH circuit units 203a and the second SH circuit unit 203b are input to a first multiplexor 204a and a second multiplexor 204b each of which outputs the electrical signals as a serial electrical signal. The first multiplexor 204a and the second multiplexor 204b include switches MSN₁ to MSN₄, MSN₅ to MSN₈, MSS₁ to MSS₄, and MSS₅ to MSS₈ for the respective signal wires. By sequentially selecting the switches MSN and MSS by the multiplexing control signal 117 from the control unit 110, an operation of converting parallel signals into a serial signal is performed. The converted serial electrical signals are input to SFN and SFS of a first output buffer 207a and a second output buffer 207b each of which impedance-converts the serial electrical signal and outputs it. The second power supply unit 107b inputs a reference voltage Vref2 to the gates of the first output buffer 207a and the second output buffer 207b via switches SRN and SRS. The switches SRN and SRS reset the inputs of a first variable amplifier 205a and a second variable amplifier 205b at predetermined timings. The electrical signals output from the first output buffer 207a and the second output buffer 207b are input to the first variable amplifier 205a and the second variable amplifier 205b. A first D/A converter 206a and a second D/A converter 206b add arbitrary offsets to the first variable amplifier 205a and the second variable amplifier 205b.

The electrical signal output from the variable amplifier 205 is input to the A/D converter 104 as the analog signal 113 output from the readout circuit 103. The third power supply unit 107c inputs a reference voltage Vref3a to the first A/D converter 104a to which the analog signal 113 output from the first readout circuit 103a is input. The third power supply unit 107c inputs a reference voltage Vref3b to the second A/D converter 104b to which the analog signal 113 output from the second readout circuit 103b is input. The reference voltage Vref3a and the reference voltage Vref3b may have the same value or values different from each other.

The non-linearity of the conversion characteristic of the A/D converter will be described here. The non-linearity represents how much the actual relationship between an analog input and a digital output deviates from an ideal line. More specifically, the non-linearity is represented by differential non-linearity (DNL) or integral non-linearity (INL). INL means a deviation of an actual input/output characteristic from an ideal input/output line upon looking over the entire input/output characteristic of the A/D converter. DNL means a deviation from an ideal step when individually observing the steps of input/output.

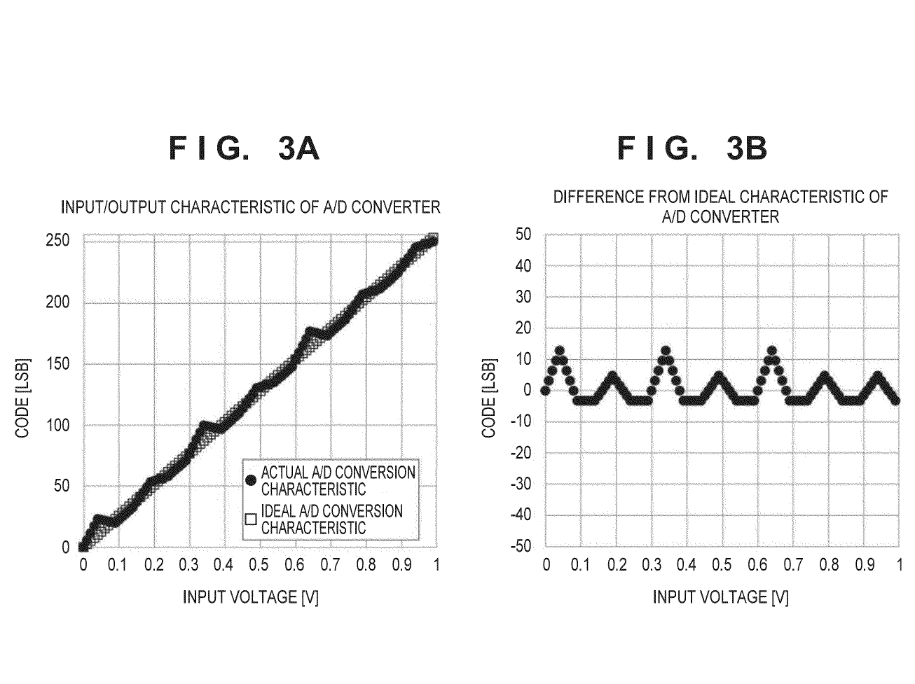

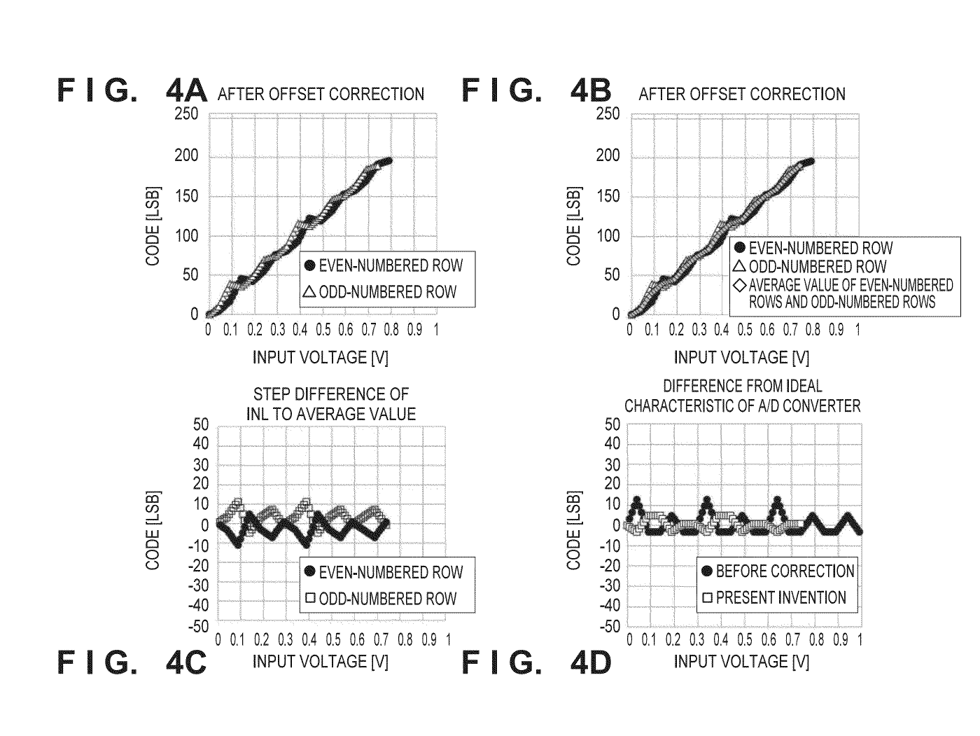

The conversion characteristic between the analog signal 112 input to the signal processing unit 106 and the output image signal 115 according to this embodiment will be described next with reference to FIGS. 3A, 3B, and 4A to 4D. A method of correcting INL according to this embodiment will be described. The influence of the non-linearity of the conversion characteristic of the A/D converter 104 included in the signal processing unit 106 will be described first with reference to FIGS. 3A and 3B. FIG. 3A shows the conversion characteristic of the A/D converter 104. Referring to FIG. 3A, the abscissa represents an input voltage input to the A/D converter 104, and the ordinate represents a digital value (code) output from the A/D converter 104. In FIG. 3A, an ideal digital signal obtained when each of 100 input voltages ranging from 0 V to 0.99 V at an interval of 0.01 V is input to the A/D converter 104 is represented by □, and an actual digital signal corresponding to the same input is represented by . Note that FIG. 3A assumes an A/D converter having a resolution of 8 bits for the sake of simplicity. The actual conversion characteristic of the A/D converter 104 has non-linearity deviated from the ideal linear conversion characteristic of the A/D converter.

FIG. 3B shows the difference of the non-linearity of the conversion characteristic of the A/D converter 104 shown in FIG. 3A from the ideal conversion characteristic. As is apparent from FIG. 3B, the difference between the digital signal output from the A/D converter 104 and the digital signal output from the A/D converter with the ideal characteristic for each input voltage is about −5 LSB to +15 LSB. For example, if parallel processing is performed using a plurality of A/D converters to do high-speed processing, a step difference caused by the non-linearity of the conversion characteristic may occur in a captured image generated based on the digital signals output from the A/D converters. In addition, for example, if a distribution is formed in offset components (to be described later) in the plane of a captured image, a partial step difference caused by the non-linearity of the conversion characteristic may occur after offset correction. As described above, the non-linearity of the A/D converter 104 may have an adverse effect on, for example, radiation image diagnosis.

A correction method in the signal processing unit 106 will be described next with reference to FIGS. 4A to 4D. FIGS. 4A to 4D are graphs for explaining a method of correcting the non-linearity of the A/D converter 104, that is, INL caused by the non-linearity using the arrangement of the radiation imaging apparatus 100 according to this embodiment and the effect thereof. Under the control of the offset generation unit 301, the control unit 110 sequentially adds an offset value that changes on a row basis to the analog signal 112 input to the conversion unit 300 out of the signal processing unit 106 and performs A/D conversion. These offset values are stored in, for example, the storage unit 109. As a result, the digital signals 114 including offset components of different values are output. The digital signals 114 output at this time have a step difference caused by the non-linearity of the conversion characteristic of the A/D converter. FIG. 4A shows the input/output characteristic of the A/D converter 104 when the offset component is changed by adding an offset value on a row basis. The abscissa represents the input voltage before the offset values are added, and the ordinate represents the code of a digital signal after offset correction of reducing offset components is performed for the A/D-converted digital signal. FIG. 4A shows a case in which the offset components are removed by offset correction. For example, an offset value of 0.2 V is set for the even-numbered rows, and an offset value of 0.25 V is set for the odd-numbered rows, thereby outputting digital signals including offset components of different values. In FIG. 4A, digital signals for input voltages of various values supplied from the pixels of the even-numbered rows are represented by , and digital signals for input voltages of various values supplied from the pixels of the odd-numbered rows are represented by Δ. After an offset value of 0.2 V is added, the input voltage supplied from the pixels of an even-numbered row is input to the A/D converter 104 and converted into a digital signal. Hence, the graph represented by in FIG. 4A is obtained by shifting the graph represented by □ in FIG. 3A such that a point in a case in which the input voltage is 0.2 V is placed on the origin. Similarly, the graph represented by Δ in FIG. 4A is obtained by shifting the graph represented by □ in FIG. 3A such that a point in a case in which the input voltage is 0.25 V is placed on the origin. As described above, when different offsets are set for the even-numbered rows and the odd-numbered rows to change the values of the offset components included in the digital signal, a lateral stripe-shaped step difference is generated on a row basis by the non-linearity that changes between the even-numbered rows and the odd-numbered rows. Meanwhile, the conversion characteristic of the A/D converter 104 exhibits non-linearity that changes between the even-numbered rows and the odd-numbered rows, as shown in FIG. 4A.

Next, processing of reducing the influence of the conversion characteristic of the A/D converter is performed for the digital signal 114 including the offset components, which is output from the conversion unit 300 and input to the digital signal processing unit 105. The correction value calculation unit 302 calculates a correction value used for correction from the input digital signal. In FIG. 4B, the average values of the input/output characteristics of analog signals of the even-numbered rows and the odd-numbered rows, for which different offset values are set, are represented by ⋄. As compared to the input/output characteristic of the A/D converter 104 shown in FIG. 3A, when the offset value that changes depending on the row is set, and the average value of the input/output characteristics of different offset values is obtained, the input/output characteristic becomes close to the ideal linear input/output characteristic, as can be seen. Next, the difference of the output value with respect to each input value between each conversion characteristic of the even-numbered rows and the odd-numbered rows and the average conversion characteristic that is the obtained average value is calculated as the correction value. FIG. 4C shows correction values calculated for the even-numbered rows and the odd-numbered rows. Next, using the calculated correction values, the correction unit 303 removes the correction value from each output value of the even-numbered rows and the odd-numbered rows, thereby performing correction. FIG. 4D shows the effect of the INL reducing method by the arrangement according to this embodiment. The A/D converter 104 before correction represented by in FIGS. 3B and 4D has a deviation of about −5 LSB to +15 LSB from the ideal characteristic. Meanwhile, when correction is performed using the arrangement according to the embodiment, the difference between the conversion characteristic and the ideal conversion characteristic after correction is about −5 LSB to +5 LSB, as indicated by □ in FIG. 4D. As is apparent, when correction is performed using the arrangement according to the embodiment, the influence of the non-linearity of the conversion characteristic of the A/D converter 104 is reduced.

In this embodiment, under the control of the control unit 110 using the offset generation unit 301, a different offset value is set on a row basis, and a step difference caused by the non-linearity of the conversion characteristic is shifted on a row basis, thereby generating different non-linearity of the A/D converter 104 on a row basis. Next, the correction value calculation unit 302 calculates the average conversion characteristic of the A/D converter 104 obtained by adding different offset values, and calculates the correction value that is the difference from the average conversion characteristic of the A/D converter 104 for each of the even-numbered rows and the odd-numbered rows. Subsequently, the correction unit 303 corrects the output digital signal using the correction value calculated by the correction value calculation unit 302. This makes it possible to improve the non-linearity of the conversion characteristic of the signal processing unit 106 between the input analog signal 112 and the output image signal 115.

The operation of the radiation imaging apparatus 100 according to this embodiment using the offset generation unit 301, the correction value calculation unit 302, and the correction unit 303 in the above-described correction of the conversion characteristic of the signal processing unit 106 will be described next in detail.

The operation of the offset generation unit 301 will be described first. The offset generation unit 301 of the control unit 110 causes the conversion unit 300 to output the digital signal 114 including an offset component of a value that periodically changes on a row basis in response to the analog signal 113 input to the conversion unit 300. In this embodiment, to generate the digital signal including a different offset value, the offset generation unit 301 performs at least one of following processes.

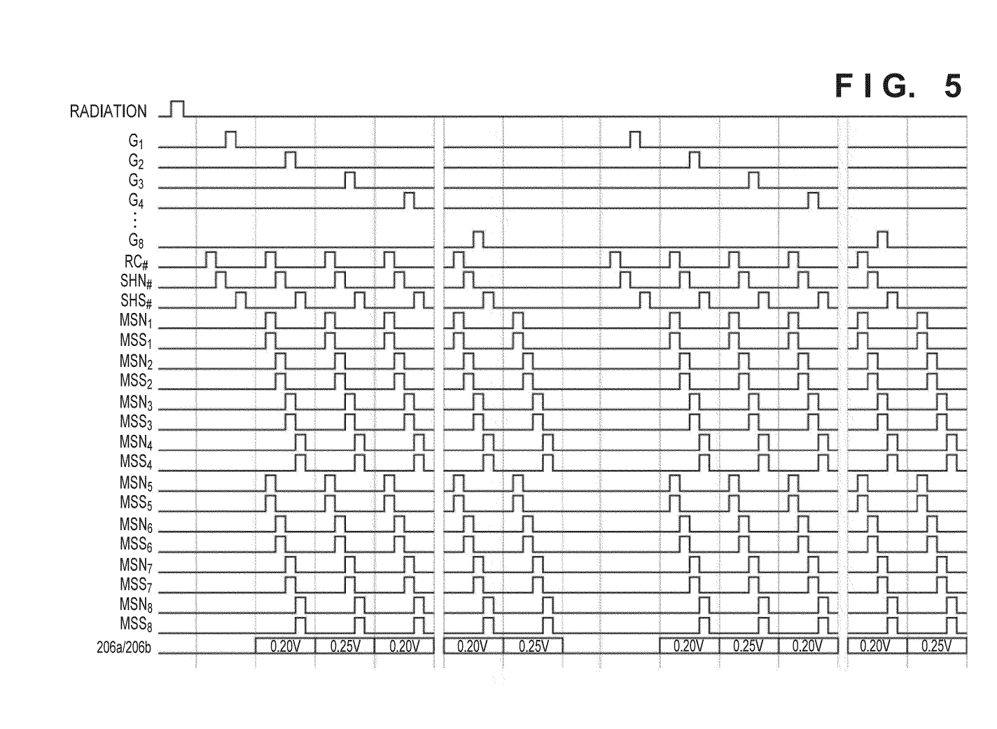

As a first process, the offset generation unit 301 may control the first D/A converter 206a and the second D/A converter 206b by the setting signal 121 such that the value of the input analog signal 113 shifts the A/D conversion characteristics of the first A/D converter 104a and the second A/D converter 104b on a row basis. More specifically, the setting is sequentially changed between the even-numbered rows and the odd-numbered rows such that, for example, the first D/A converter 206a and the second D/A converter 206b set 0.2 V in the A/D conversion operation of the first row, 0.25 V in the A/D conversion operation of the second row, and 0.2 V in the A/D conversion operation of the third row. An offset component of a value that periodically changes on a row basis is thus added to the digital signal output in response to the input analog signal. FIG. 5 is a driving timing chart in this case. Sequentially from the upper side, FIG. 5 shows incidence of radiation, the control signals of the driving wires G in the row direction, the reset switch RC, the noise sampling switch SHN, the signal sampling switch SHS, and the switches MSN and MSS of the multiplexor 204, and the set voltage of the D/A converter 206. Radiation enters at Hi level. Each control signal changes to the ON state at Hi level, and changes to the OFF state at Low level.

By the processing of the first half of the timing chart of FIG. 5, the digital signal 114 including a component according to the radiation to each pixel is supplied to the digital signal processing unit 105. An image obtained by this processing will be referred to as a radiation image hereinafter. By the processing of the second half of the timing chart of FIG. 5, the digital signal 114 including a component according to noise generated in each pixel is supplied to the digital signal processing unit 105. An image obtained by this processing will be referred to as a noise image hereinafter. The control unit 110 performs acquisition of the radiation image and acquisition of the noise image under the same setting. As a result, the two digital signals 114 concerning the same pixel have offset components of the same value. The digital signal processing unit 105 performs correction by calculating the difference between the two digital signals 114, thereby removing the offset components from the digital signals 114 and reducing the offset components. With this operation, it is possible to leave the component converted from the analog signal 113 and the step difference of INL caused by the non-linearity of the A/D converter 104 in the digital signal 114. The noise image may be acquired every time the radiation image is acquired. In addition, for example, the noise image may be acquired in advance and stored in the radiation imaging apparatus 100.

As a second process, the offset generation unit 301 may control the gains of the variable amplifiers 205a and 205b. The setting is sequentially changed between the even-numbered rows and the odd-numbered rows such that, for example, a gain=×1.00 is set in the A/D conversion operation of the first row, a gain=×1.01 is set in the A/D conversion operation of the second row, and a gain=×1.00 is set in the A/D conversion operation of the third row.

As a third process, the gains of the amplification circuit units 202a and 202b may be controlled by causing the offset generation unit 301 to adjust the gain adjusting signal 116. The setting is sequentially changed between the even-numbered rows and the odd-numbered rows such that, for example, a gain=×1.00 is set in the sample hold operation of the first row, a gain=×1.01 is set in the sample hold operation of the second row, and a gain=×1.00 is set in the sample hold operation of the third row.

As a fourth process, the values of the reference voltages Vref3a and Vref3b to be supplied by the third power supply unit 107c may be controlled by causing the offset generation unit 301 to adjust the third reference voltage adjusting signal 118c. The setting is sequentially changed between the even-numbered rows and the odd-numbered rows such that, for example, a voltage of 1.00 V is set in the A/D conversion operation of the first row, a voltage of 1.01 V is set in the A/D conversion operation of the second row, and a voltage of 1.00 V is set in the A/D conversion operation of the third row.

In the second to fourth processes, the offset components included in the digital signals 114 can be reduced by performing acquisition of the radiation image and acquisition of the noise image under the same setting, as in the first process. In this embodiment, two types of settings are alternately switched on every other row to sequentially generate the digital signals 114 including the offset components of two values. However, the setting may be changed at intervals of two or more rows, or the digital signals 114 periodically including offset components of three or more values may be generated. In this embodiment, the value of the included offset component is changed on a row basis. However, a different value may be included, for example, on a column basis.

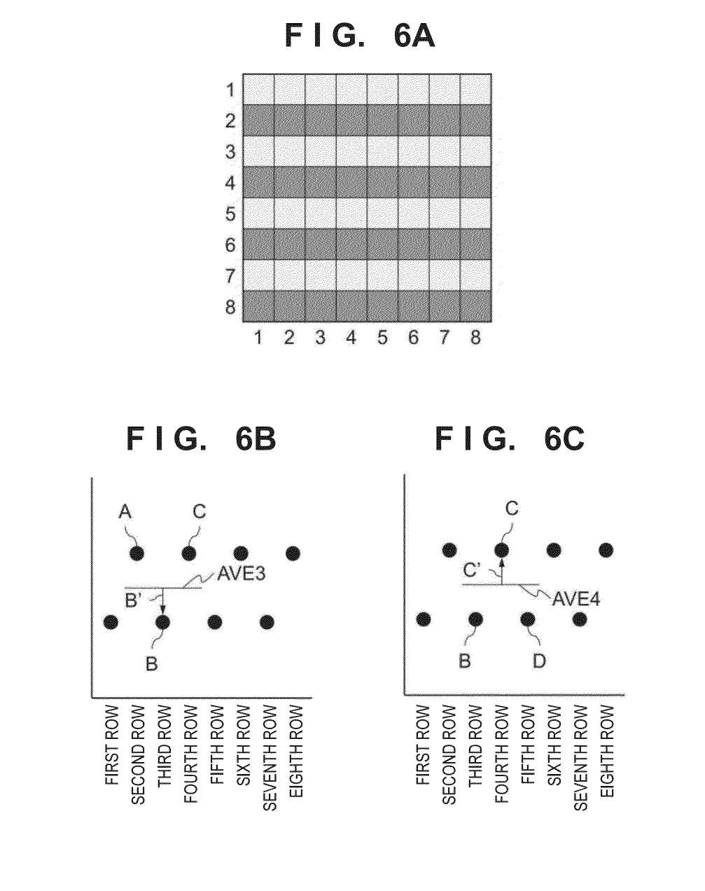

The operations of the correction value calculation unit 302 and the correction unit 303 which calculate the correction value for the step difference of INL caused by the non-linearity of the A/D converter and perform correction will be described next with reference to FIGS. 6A to 6C. As described above, conversion is performed such that the digital signal 114 output in correspondence with the analog signal 113 input to the conversion unit 300 periodically includes an offset component of a different value on a row basis under the control of the offset generation unit 301. The converted and output digital signal 114 is input to the digital signal processing unit 105.

For the digital signal 114 input to the digital signal processing unit 105, first, the above-described offset component is reduced by offset correction. FIG. 6A shows an image including information by radiation irradiation, which is generated by output signals after offset correction. The image shown in FIG. 6A is the image that has undergone only the offset correction, and is different from the image generated by the image signal 115. Even if the signal includes an offset component of a different value on a row basis, the offset component is removed by the offset correction, and only the step difference of INL caused by the non-linearity of the A/D converter 104 remains in the output signal. The odd-numbered rows of the image shown in FIG. 6A are left as white stripes, and the even-numbered rows are left as black stripes because the odd-numbered rows and the even-numbered rows have different non-linearities with respect to the ideal conversion characteristic of the A/D converter 104.

Next, the correction value calculation unit 302 calculates the correction value. For the descriptive convenience, a group formed from pixels included in at least one row or column is defined in this embodiment. Analog signals acquired from the pixels included in one group are converted into digital signals including offset components of the same value.

To calculate the correction value, the correction value calculation unit 302 obtains a representative value acquired for the pixels included in the row (in this embodiment, the third row) to be corrected, which is the first group converted into digital signals including offset components of the first value. In this embodiment, an average value B of the digital signals acquired for the pixels of the third row that is the correction row is obtained as the representative value. Note that in this embodiment, the offset components are already reduced by offset correction, as described above. In addition, the representative values of rows as the second and third groups which are adjacent before and after the correction row and are converted into digital signals including offset components of the second value different from that of the correction row are obtained. In this embodiment, an average value A of the second row and an average value C of the fourth row, which are adjacent before and after the third row, are obtained. When the average value of each row is obtained for the output signal that has undergone the offset correction, a step difference caused by the non-linearity of the A/D converter is generated on a row basis, as shown in FIGS. 6B and 6C. Next, using the obtained representative values A, B, and C of the second to fourth rows, ((A+C)/2)+B)/2 is calculated. An average value AVE3 of the representative values of the second to fourth rows is thus calculated. As the average value, not only an arithmetic mean but a weighted average may be used, as in this embodiment. After the representative values and the average value of the representative values are calculated, a correction value is calculated from these values. More specifically, B−(AVE3)=B′ is obtained, thereby calculating a correction value B′ representing the amount of the step difference of the correction row. Similarly, if the fourth row is the correction row, (((B+D)/2)+C)/2 is calculated to calculate an average value AVE4 of the representative values of the third to fifth rows which are adjacent to the correction row and are converted into digital signals including offset components of a different value. Next, C−(AVE4)=C′ is obtained, thereby calculating a correction value C′ for the fourth row.

In this manner, the correction value is calculated using the representative value of the group to be corrected and the representative value of the group converted into digital signals including offset components of a value different from that of the group to be corrected. In this embodiment, the correction value is calculated using the rows that are adjacent before and after the group to be corrected and include offset components different from those of the group to be corrected. This makes it possible to accurately extract the step difference of INL even if the captured image has the object pattern.

Particularly, in an indirect conversion type radiation imaging apparatus, high-frequency components that change between the even-numbered rows/odd-numbered rows adjacent to each other are assumed to be limited because the resolution lowers due to a wavelength converter such as a scintillator. For this reason, the step difference of INL caused by the non-linearity of the A/D converter 104 can accurately be extracted. When obtaining an average value as the representative value of each group, averaging is performed using not all pixels in the group but pixels in a number hardly influenced by random noise. Additionally, for example, when adding a plurality of types of offset components, not the average value of each group but the median value of each group may be used as the representative value.

Next, the correction unit 303 corrects the digital signals acquired for the pixels included in the correction row using the correction value calculated by the correction value calculation unit 302. As the correction, addition and/or subtraction processing is performed for the value of each digital signal using the correction value. In this embodiment, correction is performed by subtracting the correction value from the value of the acquired digital signal of each pixel. When not complex calculation processing but simple addition and/or subtraction processing is used as the correction processing, correction can be done without lowering the readout speed.

For the step difference amount of INL, an upper limit is often defined as the characteristic of the A/D converter 104 to be used. For this reason, the correction amount used when performing correction may have an upper limit by this definition to prevent overcorrection. For example, if the value calculated by the correction value calculation unit 302 is larger than the upper limit of the correction amount, correction may be performed using the upper limit of the correction amount. In this embodiment, correction value calculation and correction are performed for the image offset-corrected from the digital signals 114. However, for example, gain correction may be performed after offset correction, and after that, the correction value may be calculated to perform correction. Alternatively, for example, correction may be performed by calculating the correction value for the digital signals 114 before offset correction.

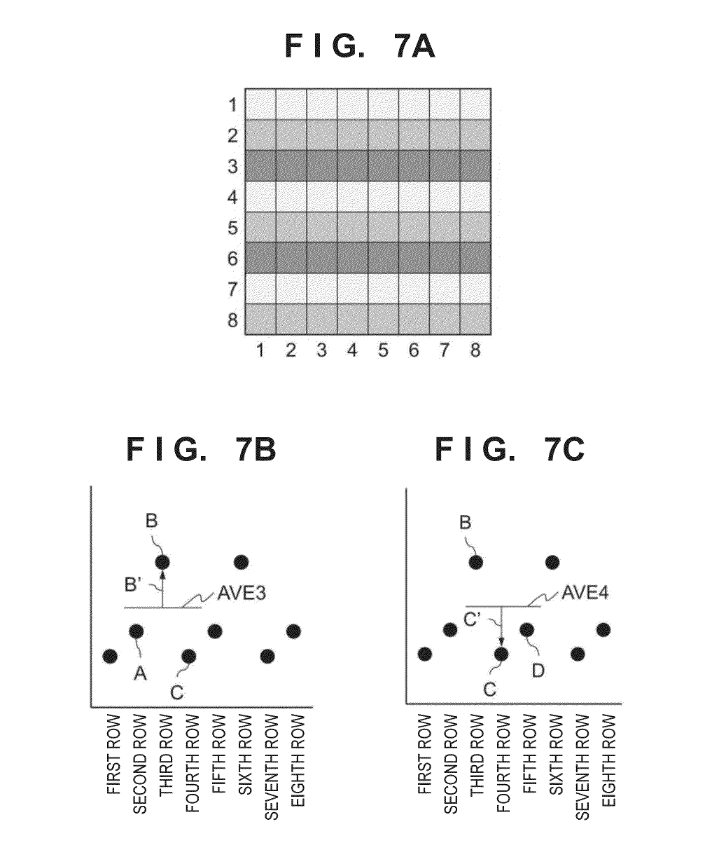

Correction in a case in which the digital signals 114 are converted into digital signals including offset components of two types, first and second values has been described with reference to FIGS. 6A to 6C. A case in which digital signals including offset components of three types of values is corrected will be described with reference to FIGS. 7A to 7C. Like FIGS. 6A to 6C, FIG. 7A shows an image generated by output signals after offset correction, and FIGS. 7B and 7C are graphs showing average values as the representative values of groups. If calculating the correction value of the third row, the average value of the representative values of the groups is obtained as (A+B+C)/3=(AVE3), and the correction value of the third row as the correction row is calculated as B−(AVE3)=B′. When three types of values are used as the offset components in this manner, the accuracy of the average value of the non-linearity of the A/D converter 104 can be improved as compared to the case in which two types of values are used. This can make the non-linearity of the A/D converter 104 to the ideal characteristic of the A/D converter. Note that the types of the values of offset components are not limited to the above-described two types or three types and may be four or more types.

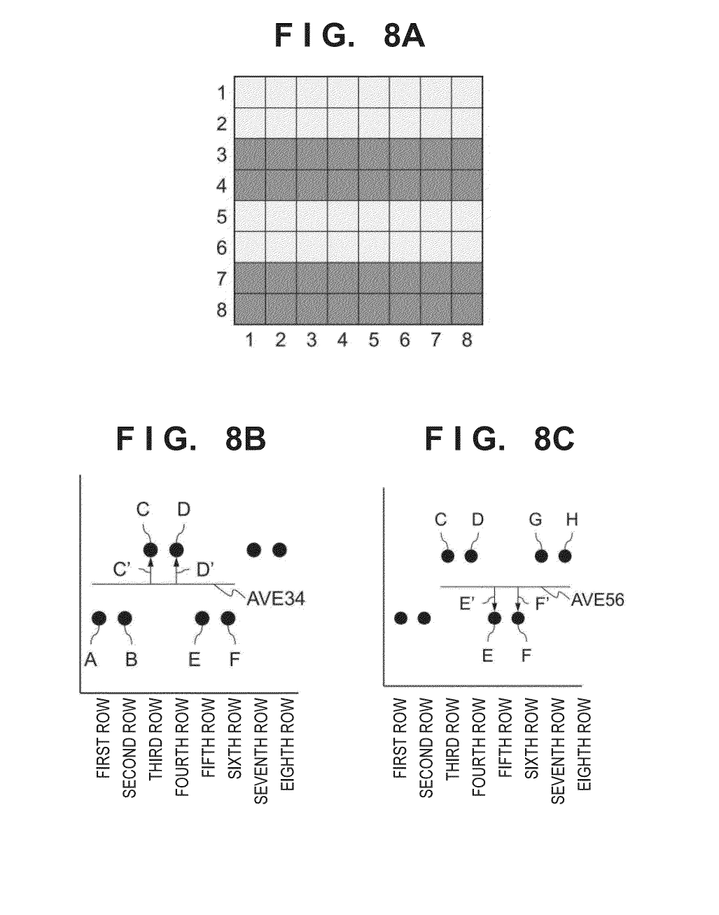

A case in which one group is formed from one row has been described with reference to FIGS. 6A to 6C and FIGS. 7A to 7C. However, a group may be formed from the pixels of two or more adjacent rows converted into digital signals including offset components of the same value. Correction in a case in which signals are converted into digital signals including offset components of two types of values at intervals of two rows will be described with reference to FIGS. 8A to 8C. Like FIGS. 6A to 6C and FIGS. 7A to 7C, FIG. 8A shows an image generated by output signals after offset correction, and FIGS. 8B and 8C are graphs showing average values as the representative values of rows. In this embodiment, the third and fourth rows that are adjacent to each other and include the same offset component are put into one group, and a correction value is calculated. In this case, the average value of the representative values of the groups is obtained as {(C+D)/2+(A+B+E+F)/4}/2=(AVE34). Next, the correction value of the third row out of the first group is calculated as C−(AVE34)=C′, and the correction value of the fourth row is calculated as D−(AVE34)=D′. If the object image includes many high-frequency components, the value of the offset component is thus changed at a long period of, for example, two rows to prevent the period from overlapping that of the object image, thereby more accurately correcting the step difference of INL. One group may be formed from three or more rows, and the value of the offset component may be changed at intervals of three or more rows. In the description of FIGS. 6A to 8C, the setting of the offset component is changed on a row basis. However, the setting may be changed on a column basis. In addition, for example, a group formed from the pixels of a plurality of rows may be converted into digital signals including offset components of three or more types of values.

In this embodiment, correction can be performed for degradation in an image caused by the non-linearity of the conversion characteristic of the A/D converter by a simple arrangement and simple processing. The method is applicable to parallel processing using a plurality of A/D converters. Since correction can be done without lowering the readout speed, the method may be suitable for a radiation imaging apparatus for moving image capturing.

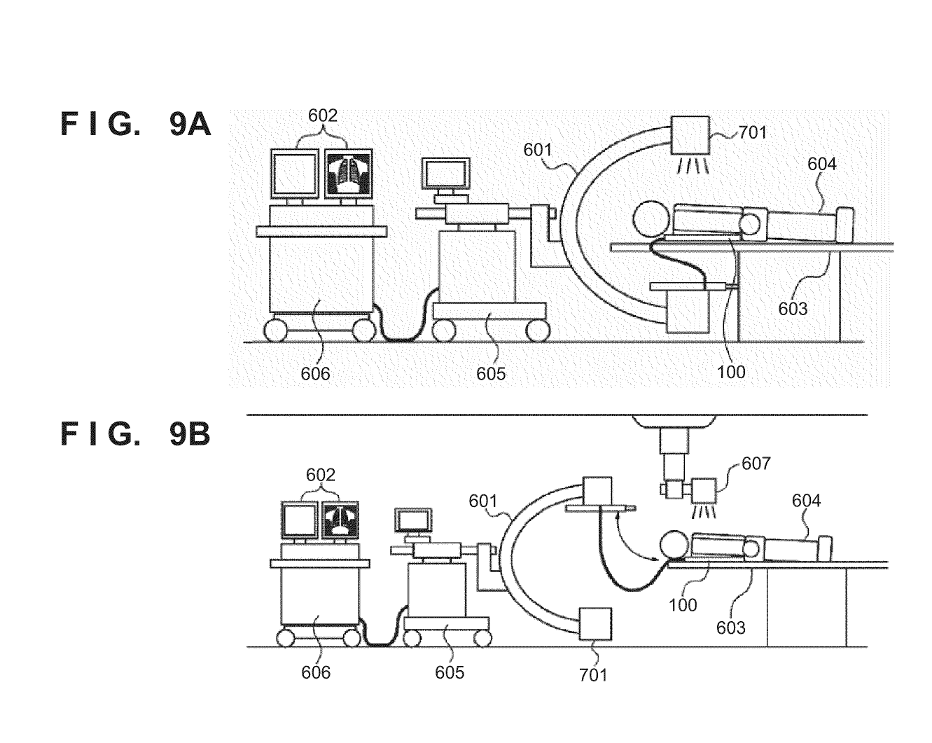

An example of application to a movable radiation imaging system using the radiation imaging apparatus 100 according to this embodiment will be described below with reference to FIGS. 9A and 9B. FIG. 9A is a conceptual view of a radiation imaging system using the portable radiation imaging apparatus 100 capable of fluoroscopy and still image capturing. FIG. 9A shows an example in which the radiation imaging apparatus 100 is detached from a C-arm 601, and imaging is performed using a radiation generating apparatus 701 provided on the C-arm 601. The C-arm 601 holds the radiation generating apparatus 701 and the radiation imaging apparatus 100. Reference numeral 602 denotes a display unit capable of displaying an image signal obtained by the radiation imaging apparatus 100; and 603, a bed used to place a subject 604. Reference numeral 605 denotes a carriage capable of moving the radiation generating apparatus 701, the radiation imaging apparatus 100, and the C-arm 601; and 606, a movable control apparatus having an arrangement capable of controlling them. The control apparatus 606 can also perform image processing of an image signal obtained by the radiation imaging apparatus 100 and transmit the image signal to the display unit 602 or the like. Image data generated by image processing of the control apparatus 606 can be transferred to a remote site by a transmission processing unit such as a telephone line. This makes it possible to display the image data on a display or save it in a recording medium such as an optical disk in another place such as a doctor room and allow a doctor at the remote site to make a diagnosis. The transmitted image data can also be recorded as a film by a film processor. Note that some or all components of the control circuit 108 according to this embodiment may be provided in the radiation imaging apparatus 100 or in the control apparatus 606.

FIG. 9B shows a radiation imaging system using the portable radiation imaging apparatus 100 capable of fluoroscopy and still image capturing. FIG. 9B shows an example in which the radiation imaging apparatus 100 is detached from the C-arm 601, and imaging is performed using a radiation generating apparatus 607 different from the radiation generating apparatus 701 provided on the C-arm 601. Note that the control circuit 108 according to this embodiment can control not only the radiation generating apparatus 701 but also the other radiation generating apparatus 607, as a matter of course.

Note that the embodiment of the present invention can be implemented when, for example, a computer executes a program. A unit for supplying the program to the computer, for example, a computer-readable recording medium such as a CD-ROM that records the program or a transmission medium such as the Internet that transmits the program can also be applied as the embodiment of the present invention. The above-described program can also be applied as the embodiment of the present invention. The program, the recording medium, the transmission medium, and a program product are incorporated in the present invention. An invention according to a combination easily anticipated from the embodiment is also incorporated in the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-058292, filed Mar. 20, 2015, which is hereby incorporated by reference wherein in its entirety.

What is claimed is:

1. A radiation imaging apparatus comprising:
a plurality of pixels arranged in a matrix and configured to detect radiation; and
a signal processing unit configured to read out an analog signal from each pixel and output an image signal,
wherein the signal processing unit comprises a conversion unit configured to convert the analog signal from each pixel into a digital signal using an A/D converter such that the digital signals of pixels included in a first group include offset components of a first value and the digital signals of pixels included in a second group include offset components of a second value different from the first value, and a digital signal processing unit configured to process the digital signal and output the image signal, and
wherein the digital signal processing unit calculates a correction value using the digital signals of the pixels included in the first group and the digital signals of the pixels included in the second group, and performs correction of reducing an influence caused by a conversion characteristic of the A/D converter in the digital signals of the pixels included in the first group using the correction value, thereby generating the image signal.

2. The apparatus according to claim 1, wherein the first group is formed from, out of the plurality of pixels, pixels included in at least one row or column, and
the second group is formed from, out of the plurality of pixels, pixels included in at least one row or column different from the first group.

3. The apparatus according to claim 1, wherein the correction value is calculated based on
a first representative value calculated from the digital signal of at least one pixel included in the first group, and
a second representative value calculated from the digital signal of at least one pixel included in the second group.

4. The apparatus according to claim 3, wherein the correction value is calculated based on
the first representative value, and
an average value of the first representative value and the second representative value.

5. The apparatus according to claim 4, wherein the correction value is a difference between the first representative value and the average value.

6. The apparatus according to claim 3, wherein the first representative value is an average value of the digital signals of at least two pixels included in the first group, and the second representative value is an average value of the digital signals of at least two pixels included in the second group.

7. The apparatus according to claim 3, wherein the first representative value is a median value of the digital signals of at least two pixels included in the first group, and the second representative value is a median value of the digital signals of at least two pixels included in the second group.

8. The apparatus according to claim 3, wherein the plurality of pixels further include a third group different from the first group and the second group, and
the correction value is calculated based on the first representative value, the second representative value, and a third representative value calculated from the digital signal of at least one pixel included in the third group.

9. The apparatus according to claim 8, wherein analog signals from the third group is converted into digital signals including the offset components of the second value.

10. The apparatus according to claim 8, wherein out of the first group, the second group, and the third group, analog signals from groups adjacent to each other are converted into digital signals including offset components of different values.

11. The apparatus according to claim 8, wherein analog signals from the third group is converted into digital signals including the offset components of a value different from both of the first value and the second value.

12. The apparatus according to claim 1, wherein analog signals from a plurality of groups that are formed from, out of the plurality of pixels, pixels included in at least one row or column and are arranged side by side are converted into digital signals periodically including offset components of different values.

13. The apparatus according to claim 1, wherein the digital signal processing unit performs the correction by addition and/or subtraction processing.

14. The apparatus according to claim 1, wherein when the digital signal processing unit performs the correction a correction amount has an upper limit, and if a value calculated using the digital signals of the pixels included in the first group and the digital signals of the pixels included in the second group is larger than the upper limit, the digital signal processing unit uses the correction amount of the upper limit as the correction value.

15. The apparatus according to claim 1, further comprising:
a driving circuit configured to scan the plurality of pixels;
a power supply unit configured to supply a bias to the conversion unit; and
a control unit configured to control the driving circuit, the conversion unit, and the power supply unit,
wherein the control unit causes the conversion unit to add an offset value to the analog signal, thereby converting the analog signal into the digital signal including the offset component.

16. The apparatus according to claim 15, wherein the control unit causes the power supply unit to supply different biases to the A/D converter, thereby converting the analog signals into the digital signals including the offset components of different values.

17. The apparatus according to claim 1, wherein the digital signal processing unit calculates the correction value and performs the correction after reducing the offset components of the first value included in the digital signals of the pixels included in the first group and the offset components of the second value included in the digital signals of the pixels included in the second group is performed.

18. The apparatus according to claim 1, further comprising a scintillator configured to convert the radiation into light,
wherein the pixel converts the light into the analog signal.

19. The apparatus according to claim 1, wherein the radiation imaging apparatus is an apparatus for moving image capturing.

20. A radiation imaging system comprising a radiation imaging apparatus and a radiation generating apparatus, wherein
the radiation imaging apparatus comprises a plurality of pixels arranged in a matrix and configured to detect radiation, and a signal processing unit configured to read out an analog signal from each pixel and output an image signal,
the signal processing unit comprises a conversion unit configured to convert the analog signal from each pixel into a digital signal using an A/D converter such that the digital signals of pixels included in a first group include offset components of a first value and the digital signals of pixels included in a second group include offset components of a second value different from the first value, and a digital signal processing unit configured to process the digital signal and output the image signal,
the digital signal processing unit calculates a correction value using the digital signals of the pixels included in the first group and the digital signals of the pixels included in the second group, and performs correction of reducing an influence caused by a conversion characteristic of the A/D converter in the digital signals of the pixels included in the first group using the correction value, thereby generating the image signal, and
the radiation generating apparatus is configured to generate radiation.
